SEQUENCE LISTING (27 sequences)

SEQ ID NO: 1 - protein, 82 aa, synthetic construct
  REGION 1..82: note = FAP binding protein Affilin 217990
MQIFVKTLTPEWHIIGKTITLEVEPSDTIENVKAKIQDKEGIPPDQQNLAWAGKQLEDGRTLSDYNIQGGSMLMLKLHLRAA

SEQ ID NO: 2 - protein, 82 aa, synthetic construct
  REGION 1..82: note = FAP binding protein Affilin 217966
MQIFVKTLTPEWHIIGKTITLEVEPSDTIENVKAKIQDKEGIPPDQQNLAWAGKQLEDGRTLSDYNINHGARLMLKLALRAA

SEQ ID NO: 3 - protein, 74 aa, synthetic construct
  REGION 1..74: note = FAP binding protein Affilin 217862
MQIFVWTKDYKGITLEVEPSDTIENVKAKIQDKEGIPPDQQTLTWKGKQLEDGRTLSDYNIMVWPNLWLRLRAA

SEQ ID NO: 4 - protein, 74 aa, synthetic construct
  REGION 1..74: note = FAP binding protein Affilin 217832
MQIFVWTRINKLITLEVEPSDTIENVKAKIQDKEGIPPDQQSLYWKGKQLEDGRTLSDYNINIWPNLYLSLRAA

SEQ ID NO: 5 - protein, 76 aa, synthetic construct
  REGION 1..76: note = FAP binding protein Affilin 217863
MQIFVWTEMHKRITLEVEPSDTIENVKAKIQDKEGIPPDQQHLTWKGKQLEDGRTLSDYNISEWPNLFLRLNLRAA

SEQ ID NO: 6 - protein, 76 aa, synthetic construct
  REGION 1..76: note = FAP binding protein Affilin 217917
MQIFVWTVVQKPITLEVEPSDTIENVKAKIQDKEGIPPDQQKLHWRGKQLEDGRTLSDYNIPFFPHLILMLQLRAA

SEQ ID NO: 7 - protein, 76 aa, synthetic construct
  REGION 1..76: note = FAP binding protein Affilin 217993-1
MQIFVWTITGKTITLEVEPSDTIENVKAKIQDKEGIPPDQQRLIWAGKQLEDGRTLSDYNIRSWPNLHLVLRLRAA

SEQ ID NO: 8 - protein, 76 aa, synthetic construct
  REGION 1..76: note = FAP binding protein Affilin 217895-1
MQIFVHTWTGKTITMEVEPSDTIENVKAKIQDKEGIPPDQQRLIWAGKQLEDGRTLSDYNIGPMRWPHLVLRLRAA

SEQ ID NO: 9 - protein, 76 aa, synthetic construct
  REGION 1..76: note = FAP binding protein Affilin 217993-2
MQIFVHTATGKTITLEVEPSDTIENVKAKIQDKEGIPPDQQRLIWAGKQLEDGRTLSDYNIEDFPPLHLVLRLRAA

SEQ ID NO: 10 - protein, 76 aa, synthetic construct
  REGION 1..76: note = FAP binding protein Affilin 217895-2
MQIFVWTDTGKTITLEVEPSDTIENVKAKIQDKEGIPPDQQRLIWAGKQLEDGRTLSDYNIWAPANLHLVLRLRAA

SEQ ID NO: 11 - protein, 162 aa, synthetic construct
  REGION 1..162: note = FAP binding protein Affilin 217993
MQIFVWTITGKTITLEVEPSDTIENVKAKIQDKEGIPPDQQRLIWAGKQLEDGRTLSDYNIRSWPNLHLVLRLRAAMQIFVHTATGKTITLEVEPSDTIENVKAKIQDKEGIPPDQQRLIWAGKQLEDGRTLSDYNIEDFPPLHLVLRLRAASAWSHPQFEK

SEQ ID NO: 12 - protein, 162 aa, synthetic construct
  REGION 1..162: note = FAP binding protein Affilin 217895
MQIFVHTWTGKTITMEVEPSDTIENVKAKIQDKEGIPPDQQRLIWAGKQLEDGRTLSDYNIGPMRWPHLVLRLRAAMQIFVWTDTGKTITLEVEPSDTIENVKAKIQDKEGIPPDQQRLIWAGKQLEDGRTLSDYNIWAPANLHLVLRLRAASAWSHPQFEK

SEQ ID NO: 13 - protein, 76 aa, synthetic construct
  REGION 1..76: note = ubiquitin
MQIFVKTLTGKTITLEVEPSDTIENVKAKIQDKEGIPPDQQRLIFAGKQLEDGRTLSDYNIQKESTLHLVLRLRAA

SEQ ID NO: 14 - protein, 759 aa, synthetic construct
  REGION 1..759: note = hFAP Uniprot. Q12884
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 15 - protein, 82 aa, synthetic construct
  REGION 1..82: note = FAP binding protein Affilin 219750
MQIFVKTLTRDYHRFGKTITLEVEPSDTIENVKAKIQDKEGIPPDQQRLAWAGKQLEDGRTLSDYNIHDGHTLKLKLLLRAA

SEQ ID NO: 16 - protein, 82 aa, synthetic construct
  REGION 1..82: note = FAP binding protein Affilin 220198
MQIFVKTLTEDWFVYGKTITLEVEPSDTIENVKAKIQDKEGIPPDQQRLAWAGKQLEDGRTLSDYNIQGGSMLMLKLHLRAA

SEQ ID NO: 17 - protein, 76 aa, synthetic construct
  REGION 1..76: note = FAP binding protein Affilin 219235
MQIFVYTMREKQITLEVEPSDTIENVKAKIQDKEGIPPDQQDLKWKGKQLEDGRTLSDYNIFWFPHLTLGLSLRAA

SEQ ID NO: 18 - protein, 76 aa, synthetic construct
  REGION 1..76: note = FAP binding protein Affilin 220134
MQIFVWTRRNKRITLEVEPSDTIENVKAKIQDKEGIPPDQQHLTWKGKQLEDGRTLSDYNISEWPNLFLRLNLRAA

SEQ ID NO: 19 - protein, 74 aa, synthetic construct
  REGION 1..74: note = FAP binding protein Affilin 220164
MQIFVWTDDWKRITLEVEPSDTIENVKAKIQDKEGIPPDQQVLAWRGKQLEDGRTLSDYNIMVWPNLWLRLRAA

SEQ ID NO: 20 - protein, 76 aa, synthetic construct
  REGION 1..76: note = FAP binding protein Affilin 220257-1
MQIFVWTITGKPITLEVEPSDTIENVKAKIQDKEGIPPDQQRLIWAGKQLEDGRTLSDYNIRSWPNLHLVLRLRAG

SEQ ID NO: 21 - protein, 76 aa, synthetic construct
  REGION 1..76: note = FAP binding protein Affilin 220257-2
MQIVVHTATGKTITLEVEPSDTIGHVKAKIQDTEGIPPDQQRLIWAGKQLEDGRTLSDYNIEDFPPLHLVLRLRAA

SEQ ID NO: 22 - protein, 152 aa, synthetic construct
  REGION 1..152: note = FAP binding protein Affilin 220257
MQIFVWTITGKPITLEVEPSDTIENVKAKIQDKEGIPPDQQRLIWAGKQLEDGRTLSDYNIRSWPNLHLVLRLRAGMQIVVHTATGKTITLEVEPSDTIGHVKAKIQDTEGIPPDQQRLIWAGKQLEDGRTLSDYNIEDFPPLHLVLRLRAA

SEQ ID NO: 23 - protein, 76 aa, synthetic construct
  REGION 1..76: note = FAP binding protein Affilin 220088
MQIFVWTDAWKLITLGVEPSDTIENVKAKIQDKEGIPPDQQHLYWKGKQLEDGRTLSDYNIVSWPNLYLKLGLRAA

SEQ ID NO: 24 - protein, 76 aa, synthetic construct
  REGION 1..76: note = FAP binding protein Affilin 223054
MQIFVWTMWHKKITLEVEPSDTIENVKAKIQDKEGIPPDQQFLVWKGKQLEDGRTLSDYNISQWPNLWLGLKLRAA

SEQ ID NO: 25 - protein, 76 aa, synthetic construct
  REGION 1..76: note = FAP binding protein Affilin 223019
MQIFVWTKTQKLITLEVEPSDTIENVKAKIQDKEGIPPDQQALFWKGKQLEDGRTLSDYNIWSWPNLWLELMLRAA

SEQ ID NO: 26 - protein, 74 aa, synthetic construct
  REGION 1..74: note = FAP binding protein Affilin 223078
MQIFVWTDDWKRIILEVEPSDTIENVKAKIQDKEGIPPDQQVLAWRGKQLEDSRTLSDYNIMVWPNLWLRLRAA

SEQ ID NO: 27 - protein, 74 aa, synthetic construct
  REGION 1..74: note = FAP binding protein Affilin 223077
MQIFVWTDDWKRITLEVEPSDTIENVKAKIQDKEGIPPDQQVLAWRGKQLEDDRTLSDYNIMVWPNLWLRLRAA

(19) United States
(12) Patent Application Publication
(10) Pub. No.: US 2026/0176310 A1
(43) Pub. Date: Jun. 25, 2026

(54) SPECIFIC BINDING MOLECULES FOR FIBROBLAST ACTIVATION PROTEIN (FAP)

(71) Applicant: Navigo Proteins GmbH, Halle/Saale (DE)

(72) Inventors: Eva Bosse-Doenecke, Halle/Saale (DE); Manja Gloser-Braeunig, Halle/Saale (DE); Jonathan Lotze, Halle/Saale (DE); Anja Katzschmann, Halle/Saale (DE); Ina Coburger, Halle/Saale (DE); Heike Boecker, Halle/Saale (DE); Hanna Bobolowski, Halle/Saale (DE)

(73) Assignee: Navigo Proteins GmbH, Halle/Saale (DE)

(21) Appl. No.: 18/714,493

(22) PCT Filed: Nov. 29, 2022
(86) PCT No.: PCT/EP2022/083725

§ 371 Date: May 29, 2024

(30) Foreign Application Priority Data
Nov. 29, 2021 (EP) 21211160.3
Feb. 11, 2022 (EP) 22156353.9

(51) Int. Cl. C07K 14/435; A61K 38/00

(52) U.S. Cl. CPC C07K 14/435; A61K 38/00

(57) ABSTRACT

The present invention relates to new ubiquitin derived molecules that bind specific to Fibroblast Activation Protein (FAP). The invention further refers to FAP binding ubiquitin derived molecules (Affilin®) proteins that further comprise a diagnostically or therapeutically active component. Further aspects of the invention cover such FAP binding proteins for a use in medicine, for example, for a use in diagnosis (including imaging) or treatment of FAP related tumors.

### FIELD OF THE INVENTION

The present invention relates to new ubiquitin derived molecules that bind specific to Fibroblast Activation Protein (FAP). The invention further refers to FAP binding ubiquitin derived molecules (Affilin®) proteins that further comprise a diagnostically or therapeutically active component. Further aspects of the invention cover such FAP binding proteins for a use in medicine, for example, for a use in diagnosis (including imaging) or treatment of FAP related tumors.

### BACKGROUND OF THE INVENTION

Cancer associated fibroblasts (CAFs) interact with cancer cells. Cancer cells induce cancer associated fibroblasts activation, and cancer associated fibroblasts support tumor growth, metastasis, invasion, and immunosuppression. The protease that is expressed by activated CAFs is Fibroblast Activation Protein (FAP; also known as prolyl endopeptidase FAP, dipeptidyl peptidase FAP, integral membrane serine protease, surface-expressed protease, etc). FAP, a stromal cell surface protease, thereby influences extracellular matrix remodeling, signaling, immunosuppression, and other processes.

FAP is highly upregulated in a multitude of cancers, including almost all carcinomas for example breast, colorectal, pancreatic, lung, brain, intrahepatic bile duct, and ovarian cancers. In addition, high levels of FAP expression can be detected in some tumors that are derived from non-epithelial tissues, such as melanoma and myeloma. Overexpression of FAP promotes tumor development and metastasis. It is not or only weakly expressed on adult normal tissues. Examples are uterus, cervix, placenta, breast and skin, which show a low to moderate expression as compared to tumors. However, high expression of FAP occurs in wound healing, inflammation such as arthritis, artherosclerotic plaques, fibrosis as well as in ischemic heart tissue after myocardial Infarction. Applications of FAP in the diagnosis and treatment of various cancers were described. Several antibodies were developed as ligands for FAP, for example Sibrotuzumab. Sibrotuzumab failed a phase II clinical trial for metastatic colorectal cancer despite tumor stroma targeting properties. In addition, 8 of 26 sibrotuzumab-treated patients developed human-anti-human antibodies with a change in pharmacokinetics and reduced tumor uptake in 4 of them. Therefore, further clinical development of sibrotuzumab was halted.

High expression levels of FAP are associated with poor prognosis for patients. However, diagnosis and treatment of FAP related cancer is not adequately addressed by existing options, and as a consequence, many patients do not adequately benefit from current strategies.

Needless to say that there is an urgent need for novel strategies for diagnosis and treatment of tumors with FAP overexpression.

One objective of the present invention is the provision of molecules for specific targeting of FAP for allowing targeted diagnostic and treatment options, including imaging of FAP positive tumors e.g. by radio-diagnostic methods and FAP-targeted radiopharmaceuticals. Targeting this tumor-associated protein may offer benefit to patients with unmet need for novel diagnostic and therapeutic routes. Specific targeting of FAP suggests potentially non-toxic diagnostic and treatment approach, due to low and restricted distribution of FAP in normal tissues. Thus, binding proteins with specificity for FAP may enable effective medical options for cancer, and finally improve quality of life for patients.

The invention provides novel FAP binding molecules for new and improved strategies in the diagnosis and treatment of FAP related cancer. Further, the novel FAP binding molecules of the invention provide improved strategies in the diagnosis and treatment of cancer related to FAP overexpression.

The above-described objectives and advantages are achieved by the subject-matters of the enclosed claims. The present invention meets the needs presented above by providing examples for FAP binding proteins. The above overview does not necessarily describe all problems solved by the present invention.

### SUMMARY OF THE INVENTION

The present disclosure provides the following items 1 to 11, without being specifically limited thereto:
1. A protein comprising an amino acid sequence of at least 80% identity to any one selected from the group of SEQ ID NOs: 1-12, 15-27 wherein the protein has a specific binding affinity for human Fibroblast Activation Protein (hFAP) of less than 100 nM. In some embodiments, a protein comprising an amino acid sequence of at least 90% identity to any one selected from the group of SEQ ID NOs: 1-12, 15-27 has a specific binding affinity for hFAP of less than 50 nM.
2. The protein of item 1 wherein the protein is a multimer. The multimer is comprising of a plurality of the proteins according to item 1. The multimer is a dimer, a trimer, or a tetramer of the protein of item 1.
3. The protein according to items 1-2 wherein the protein is a fusion protein. A fusion protein is comprising the protein according to items 1-2.
4. The protein of items 1-3, wherein the protein comprises additionally at least one coupling site for the coupling of chemical moieties. Optionally, the chemical moieties are selected from any of chelators, drugs, toxins, dyes, and small molecules.
5. The protein according to any one of items 1-4, wherein the fusion protein comprises additionally at least one diagnostically active moiety. Optionally, the diagnostically active moiety is selected from a radionuclide, fluorescent protein, photosensitizer, dye, enzyme, magnetic beads, metallic beads, colloidal particles, electron-dense reagent, biotin, digoxigenin, hapten, CAR-T, or exosomes, or any combination thereof.
6. The protein of items 1-4, wherein the protein comprises additionally at least one therapeutically active moiety. Optionally, the diagnostically active moiety is selected from a monoclonal antibody or a fragment thereof, a binding protein, a receptor or receptor domain, a receptor ligand, a radionuclide, a cytotoxic compound, a cytokine, a chemokine, an enzyme, CAR-T, or exosomes, or derivatives thereof, or any combination of the above.
7. The protein of items 1-6, wherein the protein comprises additionally at least one moiety modulating pharmacokinetics. Optionally, the moiety modulating pharmacokinetics is selected from a serum albumin, an albumin-binding protein, an immunoglobulin binding protein, or an immunoglobulin or immunoglobulin fragment, a polysaccharide, an unstructured amino acid sequence comprising amino acids alanine, glycine, serine, proline, a polyethylene glycol, a sialic acid, or a transferrin.
8. The protein of items 1-7, for use in diagnosis or treatment of FAP related diseases, such as FAP related tumors.
9. A composition comprising the protein of items 1-8 for use in medicine, preferably for use in the diagnosis or treatment of FAP related diseases.
10. A method of producing the protein of items 1-8, comprising the steps of a) culturing a host cell under conditions suitable to obtain said protein and b) isolating said protein produced.
11. A method of detecting FAP comprising a sample with a protein of items 1-8, and detecting binding of FAP with a protein of items 1-8 by contacting the sample with proteins of items 1-8.

This summary does not necessarily describe all features of the present invention. Other embodiments come apparent from a review of the ensuing detailed description.

### BRIEF DESCRIPTION OF THE FIGURES

The Figures show:

FIG. 1: shows an analysis of binding of Affilin® proteins to hFAP-Fc (label-free interaction assays using SPR). hFAP was immobilized on a CM-5 chip. After fitting the data with a 1:1 langmuir model, a K_{D} value was calculated. FIG. 1A shows Affilin®-217990 (SEQ ID NO: 1) vs hFAP (K_{D}=14 nM). FIG. 1B shows Affilin®-217832 (SEQ ID NO: 4) vs hFAP (K_{D}=3 nM).

FIG. 1C shows Affilin®-217993 (SEQ ID NO: 11) vs hFAP (K_{D}=23 nM).

FIG. 2: shows an analysis of binding of Affilin®-217990 with high affinity to mFAP-Fc (label-free interaction assays using SPR). mFAP was immobilized on a CM-5 chip. After fitting the data with a 1:1 langmuir model, a K_{D} value of 90 nM was calculated.

FIG. 3, FIG. 4, FIG. 5, and FIG. 6 show the binding affinity of FAP binding proteins in serum even after 24 h incubation. K_{D} values vs hFAP-Fc were determined after 0 h serum incubation (filled circles) and after 24 h incubation at 37° C. in serum (filled triangles) (K_{D} determination by ELISA in FIG. 3-5, K_{D} determination by flow cytometry in FIG. 6). After 24 h incubation in serum, the K_{D} value showed only minor variation compared to the K_{D} value before incubation in serum, confirming the stability of the FAP specific Affilin® proteins.

FIG. 3: shows the binding affinity of FAP binding protein Affilin®-217990 (SEQ ID NO: 1) in human serum (FIG. 3A) and mouse serum (FIG. 3B).

FIG. 4: shows the binding affinity of FAP binding protein Affilin®-220257 (SEQ ID NO: 22) in human serum (FIG. 4A) and mouse serum (FIG. 4B).

FIG. 5: shows the binding affinity of FAP binding protein Affilin®-220164 (SEQ ID NO: 19) in human serum (FIG. 5A) and mouse serum (FIG. 5B).

FIG. 6: shows the binding affinity of FAP specific Affilin®-217863 (SEQ ID NO: 5) in human serum.

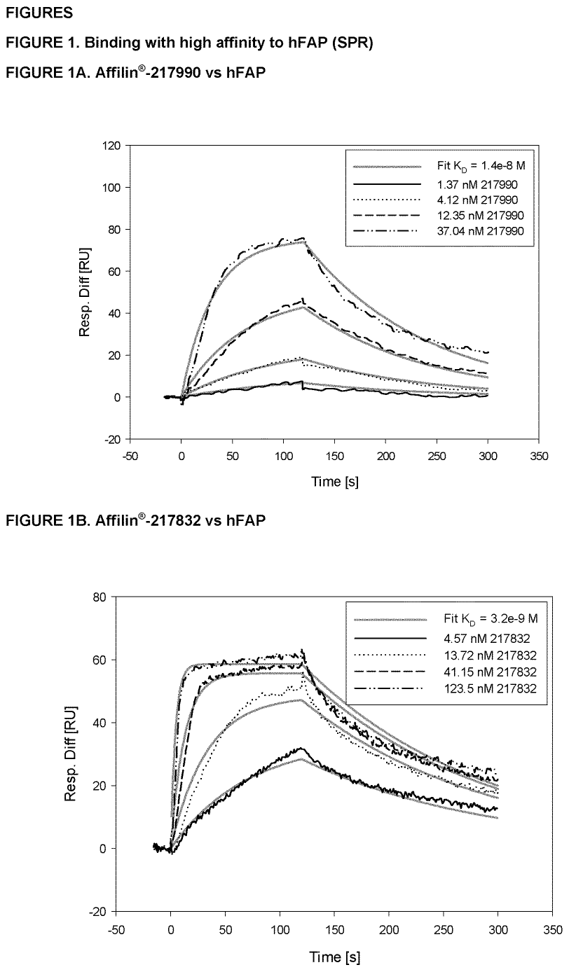

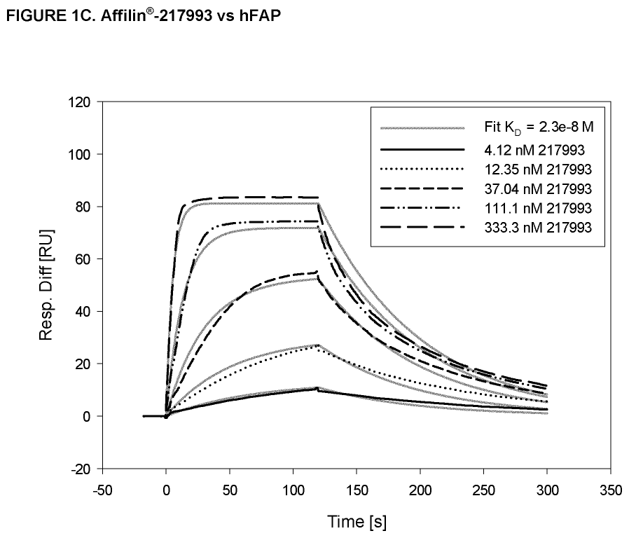

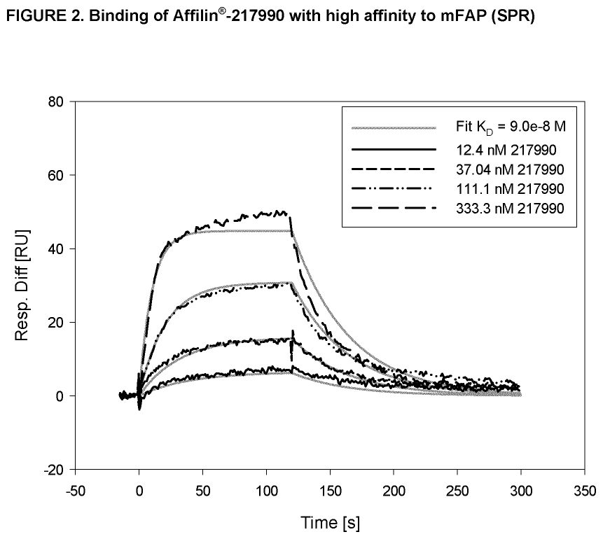

### DETAILED DESCRIPTION OF THE INVENTION

The present inventors have developed a solution to meet the strong ongoing need in the art for expanding medical options for the diagnosis and treatment of cancer by providing novel FAP binding proteins. The FAP specific proteins as defined herein are functionally characterized by high specific affinity for FAP, even on cells expressing FAP. Further, they show high levels of stability in serum (i.e. characterized by the binding affinity to FAP). The invention provides FAP binding proteins based on a ubiquitin scaffold (also known as Affilin® molecules). The FAP binding proteins as described herein thereby provide molecular formats with favorable physicochemical properties, high-level expression in bacteria, and allow easy production methods. The novel FAP binding proteins may broaden so far unmet medical strategies for the diagnosis and therapy of FAP related cancer. In particular, the FAP binding proteins may be used for imaging purposes, for example, for the presence of tumor cells expressing FAP, and for radiotherapy treatment of tumors expressing FAP, or for immunooncological treatment options.

Before the present invention is described in more detail below, it is to be understood that this invention is not limited to the particular methodology, protocols and reagents described herein as these may vary. It is also to be understood that the terminology used herein is for the purpose of describing particular aspects and embodiments only and is not intended to limit the scope of the present invention which is reflected by the appended claims. Unless defined otherwise, all technical and scientific terms used herein have the same meaning as commonly understood by one of ordinary skill in the art to which this invention belongs. This includes a skilled person working in the field of protein engineering and purification, but also including a skilled person working in the field of developing new target-specific binding molecules for use in technical applications and in therapy and diagnostics.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Throughout this specification and the claims, which follow, unless the context requires otherwise, the word “comprise”, and variants such as “comprises” and “comprising”, was understood to imply the inclusion of a stated integer or step, or group of integers or steps, but not the exclusion of any other integer or step or group of integers or steps. The term “comprise(s)” or “comprising” may encompass a limitation to “consists of” or “consisting of”, should such a limitation be necessary for any reason and to any extent.

Several documents (for example: patents, patent applications, scientific publications, manufacturer's specifications, instructions, UniProt Accession Number etc.) may be cited throughout the present specification. Nothing herein is to be construed as an admission that the invention is not entitled to antedate such disclosure by virtue of prior invention. Some of the documents cited herein may be characterized as being “incorporated by reference”. In the event of a conflict between the definitions or teachings of such incorporated references and definitions or teachings recited in the present specification, the text of the present specification takes precedence.

All sequences referred to herein are disclosed in the attached sequence listing (WIPO ST.26*. xml format) that, with its whole content and disclosure, forms part of the disclosure content of the present specification. For the avoidance of doubt, and as a precautionary measure, the WIPO ST.25-compliant sequence listing forming part of the priority application EP 22 156 353.9 is incorporated herein by reference.

### General Definitions of Important Terms Used in the Application

The term “FAP” as used herein refers to Uniprot accession number Q12884. It refers to Fibroblast Activation Protein (FAP) which is also known as prolyl endopeptidase FAP, dipeptidyl peptidas FAP, integral membrane serine protease, surface-expressed protease, etc. The term “FAP” comprises all polypeptides which show a sequence identity of at least 70%, 75%, 80%, 85%, 90%, 95%, 96% or 97% or more, or 100% to the FAP of Uniprot accession number Q12884 (human). The human FAP is 89.5% identical to mouse FAP (accession number P97321), 88.6% identical to rat FAP and 99.6% identical to cynomolgus FAP (accession number A0A2K5VGF4). The term “FAP” includes the extracellular domain (residues 26-760) of FAP. The terms “FAP binding protein” or “FAP specific Affilin® protein” or “protein comprising the FAP binding protein” are used interchangely herein and refer to a protein with high affinity binding to FAP.

The term “Affilin®” is a registered trademark of Navigo Proteins® GmbH and refers to non-immunoglobulin derived binding proteins. In the context of this invention, the term “Affilin” refers to a ubiquitin mutein.

The terms “protein” and “polypeptide” refer to any chain of two or more amino acids linked by peptide bonds, and does not refer to a specific length of the product. Thus, “peptides”, “protein”, “amino acid chain”, or any other term used to refer to a chain of two or more amino acids, are included within the definition of “polypeptide”, and the term “polypeptide” may be used instead of, or interchangeably with, any of these terms. The term “polypeptide” is also intended to refer to the products of post-translational modifications of the polypeptide, which are well known in the art.

The term “modification” or “amino acid modification” refers to a substitution, a deletion, or an insertion of a reference amino acid at a particular position in a parent polypeptide sequence by another amino acid. Given the known genetic code, and recombinant and synthetic DNA techniques, the skilled scientist can readily construct DNAs encoding the amino acid variants.

The term “ubiquitin” refers to the amino acid sequence given in SEQ ID NO: 13 and to proteins with at least 95% identity, such as for example with point mutations in positions 45, 75, 76 which do not influence binding to FAP.

The term “mutein” as used herein refers to derivatives of, for example, ubiquitin as shown in SEQ ID NO: 13, which differ from said amino acid sequence by amino acid exchanges, insertions, deletions or any combination thereof, provided that the mutein has a specific binding affinity to FAP. The FAP binding proteins of the invention are ubiquitin mutein proteins (ubiquitin muteins).

The term “substitution” is understood as exchange of an amino acid by another amino acid. The term “insertion” comprises the addition of amino acids to the original amino acid sequence.

The terms “binding affinity” and “binding activity” may be used herein interchangeably, and they refer to the ability of a polypeptide to bind to another protein, peptide, or fragment or domain thereof. Binding affinity is typically measured and reported by the equilibrium dissociation constant (K_{D}), which is used to evaluate and rank order strengths of bimolecular interactions.

The term “fusion protein” relates to a protein comprising at least a first protein joined genetically to at least a second protein. A fusion protein is created through joining of two or more genes that originally coded for separate proteins. Fusion proteins may further comprise additional domains that are not involved in binding of the target, such as but not limited to, for example, multimerization moieties, polypeptide tags, polypeptide linkers or moieties binding to a target different from FAP.

The term “amino acid sequence identity” refers to a quantitative comparison of the identity (or differences) of the amino acid sequences of two or more proteins. “Percent (%) amino acid sequence identity” with respect to a reference polypeptide sequence is defined as the percentage of amino acid residues in a sequence that are identical with the amino acid residues in the reference polypeptide sequence, after aligning the sequences and introducing gaps, if necessary, to achieve the maximum percent sequence identity. To determine the sequence identity, the sequence of a query protein is aligned to the sequence of a reference protein or polypeptide. Methods for sequence alignment are well known in the art. For example, for determining the extent of an amino acid sequence identity of an arbitrary polypeptide relative to another amino acid sequence, the SIM Local similarity program as known in the art is preferably employed. For multiple alignment analysis, Clustal Omega is preferably used, as known to someone skilled in the art.

As used herein, the phrases “percent identical” or “percent (%) amino acid sequence identity” or “percent identity”, in the context of two polypeptide sequences, refer to two or more sequences or subsequences that have in some embodiments at least 80%, in some embodiments at least 85%, in some embodiments at least 90%, in some embodiments at least 91%, some embodiments at least 92%, in some embodiments at least 93%, in some embodiments at least 94%, in some embodiments at least 95%, in some embodiments at least 96%, in some embodiments at least 97%, in some embodiments at least 98%, and in some embodiments 100% amino acid residue identity, when compared and aligned for maximum correspondence, as measured using one of the following sequence comparison algorithms or by visual inspection. For clarity reasons, for example a sequence with at least 80% identity includes all sequences with identities higher than 80% identity, e.g. embodiments with at least 80%, at least 85%, at least 90%, at least 91%, at least 92%, at least 93%, at least 94%, at least 95%, at least 96%, at least 97%, at least 98%, at least 99%, or 100% amino acid identity. For clarity reasons, for example a sequence with at least 90% identity includes all sequences with identities of 90% or higher than 90%, e.g. embodiments with at least 90%, at least 91%, at least 92%, at least 93%, at least 94%, at least 95%, at least 96%, at least 97%, at least 98%, at least 99%, or 100% amino acid identity.

The term “fused” means that polypeptide components or units are linked by peptide bonds, either directly or via peptide linkers. In various embodiments, the term “fused” may mean that polypeptide components or units are linked by a non-peptide linker, e.g., through chemical conjugation.

The term “fusion protein” relates to a protein comprising at least a first protein joined genetically to at least a second protein. A fusion protein is created through joining of two or more genes that originally coded for separate proteins. Thus, a fusion protein may comprise a multimer of identical or different proteins which are expressed as a single, linear polypeptide. In various embodiments, a fusion protein is created through joining of two or more polypeptides via a non-peptide linker, e.g., through chemical conjugation. Fusion proteins may further comprise additional domains that are not involved in binding of the target, such as but not limited to, for example, multimerization moieties, polypeptide tags, polypeptide linkers or moieties binding to a target different from FAP.

### DETAILED DESCRIPTION OF THE EMBODIMENTS OF THIS INVENTION

Structural characterization of FAP binding proteins. The FAP binding protein as described herein comprises, essentially consists, or consists of an amino acid sequence selected from any one of the group of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, or the FAP binding protein is selected from amino acid sequences with at least 80% identity thereto, respectively. In various embodiments, the FAP binding protein comprises or consists of an amino acid sequence of any one of SEQ ID NOs: 1-12, and 15-27, or an amino acid sequence with at least 80%, at least 85%, at least 90%, at least 91%, at least 92%, at least 93%, at least 94%, at least 95%, at least 96%, at least 97%, at least 98%, or at least 99% identity thereto. In various embodiments, the FAP binding protein comprises or essentially consists of or consists of an amino acid sequence of any of SEQ ID NOs: 1, 2, 3, 4, 5, 6, 7, 8, 9, 10, 11, 12, 15, 16, 17, 18, 19, 20, 21, 22, 23, 24, 25, 26, 27, or an amino acid with at least 80%, at least 85%, at least 90%, at least 91%, at least 92%, at least 93%, at least 94%, at least 95%, at least 96%, at least 97%, at least 98%, or at least 99% identity to any of SEQ ID NOs: 1-12, and 15-27.

All FAP binding proteins are based on ubiquitin and are structurally related. All FAP binding proteins as described herein share the same basic protein scaffold.

In some embodiments, modifications in ubiquitin that result in binding to FAP occur in regions corresponding to amino acid positions 6-15, and/or 42-46, and/or 62-72 of ubiquitin (SEQ ID NO: 13).

In some embodiments, the FAP specific ubiquitin muteins have an aromatic amino acid in position 64 of SEQ ID NO: 13 (E64W oder E64F).

In some embodiments, the FAP specific ubiquitin muteins have a proline (P) in position 65 of SEQ ID NO: 13 (S65P).

In some embodiments, the FAP specific ubiquitin muteins have an aromatic amino acid in position 64 of SEQ ID NO: 13 (E64W oder E64F) and a proline (P) in position 65 of SEQ ID NO: 13 (S65P).

In some embodiments, the FAP specific ubiquitin muteins have an aromatic amino acid in position 6 of SEQ ID NO: 13 (K6W oder K6Y).

In some embodiments, the FAP specific ubiquitin muteins have a basic amino acid in position 46 of SEQ ID NO: 13 (A46K oder A46R).

Some embodiments provide FAP specific ubiquitin muteins with at least 7 further substitutions in position 6, 8, 9, 10, 12, 42, 44, 45, 46, 62, 63, 66, 68, and 70 of SEQ ID NO: 13.

In some embodiments, one or two or more further substitutions in ubiquitin are provided.

In some embodiments, the FAP specific ubiquitin muteins have an additional insertion of 6 amino acids between positions corresponding to position 9 and 10 (see for example, SEQ ID NOs: 1, 2, 15, 16; Affilin®-217990, Affilin®-217966, Affilin®-219750, Affilin®-220198, respectively). In some embodiments, FAP specific ubiquitin muteins with additional insertion of 6 amino acids between positions corresponding to position 9 and 10 of SEQ ID NO: 13 (ubiquitin) have
(i) an Alanin (A) in the amino acid position that is corresponding to position 44 of SEQ ID NO: 13 (exchange I44A; corresponding to position 50 in Affilin® proteins of SEQ ID NO: 1, 2, 15, 16), and/or
(ii) a Glycin (G) in the amino acid position that is corresponding to position 64 of SEQ ID NO: 13 (exchange E64G; corresponding to position 70 in Affilin® proteins of SEQ ID NO: 1, 2, 15, 16), and/or
(iii) a Lysine (K) in the amino acid position that is corresponding to position 70 of SEQ ID NO: 13 (exchange V70K; corresponding to position 76 in Affilin® proteins of SEQ ID NO: 1, 2, 15, 16).

In some embodiments, one or two or more further substitutions in ubiquitin are provided.

In various embodiments, the FAP binding protein comprises or essentially consists of or consists of an amino acid sequence of SEQ ID NO: 1, or an amino acid with at least 80% identity, at least 85% identity, at least 90% identity to SEQ ID NO: 1. For example, SEQ ID NO: 1 (217990) is 93.9% identical to SEQ ID NO: 2 (217996) and to SEQ ID NO: 16 (220198), respectively. SEQ ID NO: 1 (217990) is 87.8% identical to SEQ ID NO: 15 (217750).

In various embodiments, the FAP binding protein comprises, or essentially consists of, or consists of, an amino acid sequence of SEQ ID NO: 4, or an amino acid with at least 80% identity, at least 85% identity, at least 90% identity to SEQ ID NO: 4.

In various embodiments, the FAP binding protein comprises or essentially consists of or consists of an amino acid sequence of SEQ ID NO: 19, or an amino acid with at least 80% identity, at least 85% identity, at least 90% identity to SEQ ID NO: 19.

Functional characterization. The FAP binding protein as described herein has a binding affinity (K_{D}) of less than 100 nM for FAP. In some embodiments, the FAP binding proteins as described herein bind human FAP with measurable binding affinity of less than 100 nM, less than 50 nM, less than 25 nM, less than 15 nM, or even less than 10 nM. The appropriate methods are known to those skilled in the art or described in the literature. The methods for determining the binding affinities are known per se and can be selected for instance from the following methods known in the art: enzyme-linked immunosorbent assay (ELISA), surface plasmon resonance (SPR), kinetic exclusion analysis (KinExA assay), Bio-layer interferometry (BLI), flow cytometry, fluorescence spectroscopy techniques, isothermal titration calorimetry (ITC), analytical ultracentrifugation, radioimmunoassay (RIA or IRMA), and enhanced chemiluminescence (ECL). Some of the methods are described in the Examples below. In some embodiments, the FAP binding proteins as described herein bind human FAP with measurable binding affinity of less than 100 nM, less than 50 nM, less than 25 nM, less than 15 nM, or even less than 10 nM, as determined by surface plasmon resonance. Typically, the dissociation constant K_{D} is determined at 20° C., 25° C., or 30° C. The lower the K_{D} value, the greater the binding affinity of the biomolecule for its binding partner. The higher the K_{D} value, the more weakly the binding partners bind to each other (see Figures and Examples). In one embodiment, the FAP binding protein has a dissociation constant K_{D} to human FAP in the range between 0.1 nM and 100 nM, preferably between 0.1 nM and 50 nM, more preferably between 0.1 nM and 25 nM, and even more preferably between 0.1 nM and 15 nM. In some embodiments, the FAP binding protein of the invention binds to human FAP and to cynomolgus FAP. In some embodiments, the FAP binding protein of the invention binds to human FAP and to mouse FAP.

Preferred embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 50 nM.

Other preferred embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 25 nM. Some preferred embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 15 nM. Some preferred embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 10 nM.

In some embodiments, the FAP binding proteins as described herein are particularly stable under different conditions, as shown in the Examples and in the Figures.

In some embodiments, the FAP binding proteins are stable in the presence of serum for at least 24 h at 37° C. In some embodiments, the FAP binding proteins are stable in the presence of human serum for at least 24 h at 37° C., as described in Examples in more detail. In some embodiments, the FAP binding proteins are stable in the presence of mouse serum for at least 24 h at 37° C. For example, the stability of a FAP binding protein can be determined by measuring the binding affinity (K_{D}) after incubation in serum for a long period of time (e.g. 24 h) at temperatures as high as 37° C. using standard methods as described above herein and in the Examples and Figures. The binding affinity for FAP can be assessed for each binding protein before and after serum incubation as described herein. Some preferred embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for hFAP of less than 5 nM, preferably less than 2 nM as determined via ELISA as described herein. Some preferred embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for hFAP of less than 5 nM, preferably less than 2 nM after incubation for 24 h at 37° C. in serum, as determined via ELISA as described herein.

A FAP binding protein that is stable in serum for a prolonged period of time at 37° C. shows no significant loss in binding affinity to FAP, reflecting an extraordinary stability of the FAP binding proteins as described herein. No significant loss means that the binding affinity is not reduced more than about 2-fold compared to the value obtained before incubation in serum.

In some embodiments, the FAP binding protein as described herein is stable at high temperatures, preferably of at least 60° C., more preferably at least 70° C. For stability analysis, for example spectroscopic or fluorescence-based methods in connection with chemical or physical unfolding are known to those skilled in the art. For example, the stability of a molecule can be determined by measuring the thermal melting (Tₘ) temperature, the temperature in ° Celsius (° C.) at which half of the molecules become unfolded, using standard methods. Typically, the higher the Tₘ, the more stable the molecule.

In some embodiments, the specific binding of the FAP specific Affilin® protein as described herein is confirmed by cellular FAP binding analysis with overexpressing cells (see Examples). Cellular FAP binding of the FAP specific Affilin® protein can be determined by standard methods, including immunofluorescence microscopy and flow cytometric analysis.

Multimers. Some embodiments relate to a protein comprising an amino acid sequence of at least 90% identity to any one selected from the group of SEQ ID NOs: 1-12, 15-27, wherein the protein has a specific binding affinity for human Fibroblast Activation Protein (hFAP) of less than 50 nM and wherein the protein is a multimer. In some embodiments, the FAP binding protein is a multimer comprising of a plurality of the FAP binding proteins as defined herein, such as an amino acid sequence of at least 90% preferably at least 94% or at least 95%, identity to any one selected from the group of SEQ ID NOs: 1-12, 15-27, wherein the protein has a specific binding affinity for human Fibroblast Activation Protein (hFAP) of less than 50 nM. A multimer may comprise two, three, four, or more FAP binding proteins. In one embodiment, the FAP binding protein comprises 2, 3, 4, or more FAP binding proteins linked to each other, i.e. the FAP-binding protein can be a dimer, trimer, or tetramer, etc. Multimers of the invention are fusion proteins generated artificially, generally by recombinant DNA technology well-known to a skilled person. A multimer may comprise two FAP binding domains, wherein said FAP binding domains preferably comprise or essentially consist of an amino acid sequence as described above. In some embodiments, the multimer is a dimer. The present invention provides dimers of amino acid sequences of SEQ ID NOs: 11, 12, 22.

In some embodiments, two or more FAP binding proteins are directly linked. In some embodiments, two or more FAP binding proteins are linked by a peptide linker. In various embodiments, two or more FAP binding proteins are linked via a peptide linker of up to 30 amino acids. In some embodiments, two FAP binding proteins are directly linked. In some embodiments, two FAP binding proteins are linked by a peptide linker.

Fusion protein. Some embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 50 nM and wherein the protein is a fusion protein. Some embodiments relate to a protein comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 50 nM and wherein the protein is a multimer of such amino acids and wherein the protein is a fusion protein.

In some embodiments, the protein of the invention is a fusion protein comprising of the FAP binding proteins as defined herein and at least a second protein. In some embodiments, the protein of the invention is a fusion protein comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 50 nM, and at least a second protein. Accordingly, some embodiments encompass fusion proteins comprising one or two or more FAP binding protein(s) thereof as disclosed herein and one or two or more further polypeptide(s).

Coupling sites. In some embodiments, the protein of the invention is comprising an amino acid sequence of at least 90%, preferably at least 94% or at least 95%, identity to any one selected from the group of SEQ ID NOs: 1-12, 15-27, wherein the protein has a specific binding affinity for human Fibroblast Activation Protein (hFAP) of less than 50 nM, or a fusion protein comprising such amino acid, and/or a multimer comprising above described amino acid sequence, and further one or more coupling site(s) for the coupling of chemical moieties. In some embodiments, the protein comprising the FAP binding protein as described herein further comprises one or more coupling site(s) for the coupling of chemical moieties. A coupling site is capable of reacting with other chemical groups to couple the FAP binding protein to chemical moieties. The defined number and defined position of coupling sites enables site-specific coupling of chemical moieties to the FAP binding proteins as described herein. Thus, a large number of chemical moieties can be bound to a FAP binding protein if required. The number of coupling sites can be adjusted to the optimal number for a certain application by a person skilled in the art to adjust the amount of the chemical moieties accordingly. In selected embodiments, the coupling site may be selected from the group of one or more amino acids which can be labeled with specific chemistry such as one or more cysteine residues, one or more lysine residues, one or more tyrosine, one or more tryptophan, or one or more histidine residues. The FAP binding protein may comprise 1 to 20 coupling site(s), preferably 1 to 6 coupling site(s), preferably 2 coupling sites, or preferably one coupling site.

Coupling domains. One embodiment provides a FAP binding protein that comprises at least one coupling domain of 1 to 80 amino acids comprising one or more coupling sites. In some embodiments, the coupling domain of 1 to 80 amino acids may comprise alanine, proline, or serine, and as coupling site cysteine. n other embodiments, the coupling domain of 5 to 80 amino acids may consist of alanine, proline, serine, and as coupling site cysteine. In one embodiment, the coupling domain is consisting of 20-60% alanine, 20-40% proline, 10-60% serine, and one or more cysteine as coupling site(s) at the C- or N-terminal end of the FAP binding protein as described herein. In some embodiments the amino acids alanine, proline, and serine are randomly distributed throughout a coupling domain amino acid sequence so that not more than a maximum of 2, 3, 4, or 5 identical amino acid residues are adjacent, preferably a maximum of 3 amino acids. The composition of the 1 to 20 coupling domains can be different or identical.

In some embodiments, the chemical moieties are selected from any of chelators, drugs, toxins, dyes, and small molecules. In some embodiments, at least one of the chemical moieties is a chelator designed as a complexing agent for coupling one or more further moieties to the targeted compound to the FAP binding protein as disclosed herein. One embodiment relates to a FAP binding protein wherein the chelator is a complexing agent for coupling one or more radioisotopes or other detectable labels.

Diagnostic moiety. Various embodiments relate to the FAP binding protein as described herein or a fusion protein comprising the FAP binding protein as described herein and a diagnostic moiety. Various embodiments relate to the FAP binding protein as described herein and at least one diagnostic moiety. Various embodiments relate to the FAP binding protein as described herein and a diagnostic moiety. Various embodiments relate to a fusion protein wherein the fusion protein comprises the FAP binding protein as described herein and a diagnostic moiety (i.e. at least one diagnostic moiety).

In some embodiments, such diagnostic moiety may be selected from radionuclides, fluorescent proteins, photosensitizers, dyes, enzymes, magnetic beads, metallic beads, colloidal particles, electron-dense reagent, biotin, digoxigenin, hapten, or any combination of the above.

In some embodiments, the FAP binding protein (including the fusion protein as described herein) as described above can be employed, for example, as imaging agents, for example to evaluate presence of tumor cells or metastases, tumor distribution, and/or recurrence of tumor. Methods for detection or monitoring of cancer cells involve imaging methods. Such method of imaging cancer cells comprise the proteins comprising the FAP binding protein herein (including the fusion protein as described herein) conjugated to a diagnostic moiety for imaging FAP related cancer cells by, for example, radioimaging or photoluminescense or fluorescence. In some embodiments, the method for imaging (detecting) cancer cells comprise the FAP binding protein as described (including the fusion protein as described herein) herein conjugated or coupled to a diagnostic moiety for imaging FAP related tumor cells, for example a radionuclide or a fluorescent protein.

Therapeutic moiety. Various embodiments relate to the FAP binding protein as described herein or a fusion protein comprising the FAP binding protein as described herein and a therapeutic moiety. Various embodiments relate to the FAP binding protein as described herein and at least one therapeutic moiety. Various embodiments relate to the FAP binding protein as described herein and a therapeutic moiety. Various embodiments relate to a fusion protein wherein the fusion protein comprises the FAP binding protein as described herein and a therapeutic moiety (i.e. at least one therapeutic moiety).

In some embodiments, such therapeutically active moiety may be selected from a monoclonal antibody or a fragment thereof, an extracellular domain of a receptor or fragments thereof, a binding protein, a radionuclide, a cytotoxic compound, a cytokine, a chemokine, an enzyme, CAR-T, or exosomes, or derivatives thereof, or any combination of the above.

In some embodiments, the FAP binding protein as described herein or a fusion protein as described herein comprising a therapeutically active component is used methods of targeted delivery of any of the above listed components to the FAP expressing tumor cells. Thereby, the FAP specific binding protein accumulate in FAP expressing tumor cells, and as result of the high specificity for tumor cells, only low levels of toxicity to normal cells are expected. In some embodiments, the FAP binding protein as described herein (including fusion proteins) are conjugated or coupled or joined to a therapeutic moiety as described herein.

Radionuclides. Suitable radionuclides for applications in imaging (for example, in vitro) or for methods of treatment involving radiotherapy include for example but are not limited to the group of gamma-emitting isotopes, the group of positron emitters, the group of beta-emitters, and the group of alpha-emitters. In some embodiments, suitable conjugation partners include chelators such as 1,4,7,10-tetraazacyclododecane-1,4,7,10-tetraacetic acid (DOTA) or diethylene triamine pentaacetic acid (DTPA) or their activated derivatives, nanoparticles and liposomes. In various embodiments, DOTA may be suitable as complexing agent for radioisotopes and other agents for imaging.

Moiety modulating pharmacokinetics. Various embodiments relate to the FAP binding protein as described herein or a fusion protein comprising the FAP binding protein as described herein and a moiety modulating pharmacokinetics. Various embodiments relate to the FAP binding protein as described herein and at least one moiety modulating pharmacokinetics. Various embodiments relate to the FAP binding protein as described herein and a moiety modulating pharmacokinetics. Various embodiments relate to a fusion protein wherein the fusion protein comprises the FAP binding protein as described herein and a moiety modulating pharmacokinetics (i.e. at least one moiety modulating pharmacokinetics).

In some embodiments, the the FAP binding protein as described herein or a fusion protein comprising the FAP binding protein of the invention further comprises at least one moiety modulating pharmacokinetics wherein the moiety modulating pharmacokinetics is selected from an albumin-binding peptide, an albumin-binding protein, a polyethylene glycol, a serum albumin (e.g. mouse serum albumin or human serum albumin), an immunoglobulin binding peptide. an immunoglobulin. an immunoglobulin fragment, a sialic acid, or a transferrin, a polysaccharide (for example, hydroxylethyl starch), or an unstructured amino acid sequence which increases the hydrodynamic radius (such as a multimer comprising amino acids alanine, glycine, serine, proline). In some embodiments, the FAP binding protein as described herein or a fusion protein comprising the FAP binding protein as described herein is conjugated or coupled or fused to a moiety modulating pharmacokinetics as described above.

Some embodiments comprise a fusion protein comprising the FAP binding protein as described herein and a moiety modulating pharmacokinetics as described above. Some embodiments comprise a fusion protein comprising the FAP binding protein as described herein and an albumin-binding peptide or an albumin-binding protein, an immunoglobulin binding peptide, or an immunoglobulin or immunoglobulin fragments.

In some embodiments, the FAP binding protein as described herein or a fusion protein comprising the FAP binding protein is conjugated or coupled or fused to a diagnostic moiety and are further conjugated or coupled or joined to a moiety modulating pharmacokinetics as described above.

Some specific embodiments comprise the FAP binding protein as described herein or a fusion protein comprising the FAP binding protein as described herein and a radionuclide and a moiety modulating pharmacokinetics.

In some embodiments, the FAP binding protein as described herein or a fusion protein comprising the FAP binding protein is conjugated or coupled or fused to a therapeutic moiety and are further conjugated or coupled or joined to a moiety modulating pharmacokinetics as described above.

Some embodiments comprise the FAP binding protein as described herein or a fusion protein comprising the FAP binding protein as described herein and additionally a moiety modulating pharmacokinetics as described above and a therapeutic moiety. Some specific embodiments comprise the FAP binding protein as described herein or a fusion a protein comprising the FAP binding protein as described herein and a therapeutic moiety and a moiety modulating pharmacokinetics.

Several techniques for producing protein comprising the the FAP binding protein as described herein or a fusion protein comprising the FAP binding protein with extended half-life are known in the art, for example, direct fusions of the moiety modulating pharmacokinetics with the FAP binding protein as described herein or a fusion protein comprising the FAP binding protein as described above or chemical coupling methods. The moiety modulating pharmacokinetics can be attached for example at one or several sites of the FAP binding protein as described herein or a fusion protein comprising the FAP binding protein through a peptide linker sequence or through a coupling site as described above.

Further moieties. In some embodiments, conjugation of proteinaceous or non-proteinaceous moieties to the FAP binding protein as described herein or a fusion protein comprising the FAP binding protein may be performed applying chemical methods well-known in the art. In some embodiments, coupling chemistry specific for derivatization of cysteine or lysine residues may be applicable. Chemical coupling can be performed by chemistry well known to someone skilled in the art, including but not limited to, substitution, addition or cycloaddition or oxidation chemistry (e.g. disulfide formation).

Molecules for purification/detection. In some embodiments, additional amino acids can extend either at the N-terminal end of the FAP binding protein as described herein or a fusion protein comprising the FAP binding protein or the C-terminal end or both. Additional sequences may include for example sequences introduced e.g. for purification or detection. In one embodiment, additional amino acid sequences include one or more peptide sequences that confer an affinity to certain chromatography column materials. Typical examples for such sequences include, without being limiting, Strep-tags, oligohistidine-tags, glutathione S-transferase, maltose-binding protein, inteins, intein fragments, or the albumin-binding domain of protein G.

The FAP binding protein for use in medicine. Various embodiments relate to the FAP binding protein as described herein or a fusion protein comprising the FAP binding protein as disclosed herein for use in medicine. In one embodiment, the FAP binding protein as described herein or a fusion protein comprising the FAP binding protein is used in medicine to diagnose or treat cancer associated with FAP expression. The FAP binding protein as described herein or a fusion protein comprising the FAP binding protein as disclosed herein allow selective diagnosis and treatment of FAP related cancer cells or cancer tissues, for example, from breast, colorectal, pancreatic, lung, brain, intrahepatic bile duct, and ovarian cancers, or tumors that are derived from non-epithelial tissues, such as melanoma and myeloma. FAP binding proteins are used in diagnosis (imaging) and treatment) for most epithelial cancers, including of breast, lung, colorectal and pancreatic carcinomas. FAP is known to be upregulated in tumor cells, possibly resulting in uncontrolled growth of tumor cells and in the formation of metastases. In one embodiment, the FAP binding protein is used to diagnose FAP related tumors by applying in vitro methods.

One embodiment is a method of diagnosing (including monitoring) a subject having a FAP related tumor the method of diagnosis (including monitoring/imaging) comprising administering to the subject the FAP binding protein as described herein or a fusion protein comprising the FAP binding protein as described, optionally conjugated to radioactive molecules. In various embodiments, the FAP binding protein as described herein or a fusion protein comprising the FAP binding protein as disclosed herein may be used in methods of diagnosis of FAP related tumors, optionally wherein the FAP binding protein as described herein or a fusion protein comprising the FAP binding protein is conjugated to a radioactive molecule. In some embodiments, methods for imaging specific tissues or cells expressing FAP comprise the FAP binding protein as described herein or a fusion protein comprising the FAP binding protein as described herein. In some embodiments, the methods for imaging specific tissues or cells expressing FAP comprise labels conjugated to the FAP binding protein. In some embodiments, such labels are selected from radioactive or fluorescent molecules. In some embodiments, the methods for imaging specific tissues or cells expressing FAP comprise labels conjugated to the FAP binding protein are employed to visualize FAP on specific tissues or cells, for example, to evaluate presence of FAP related tumor cells, FAP related tumor distribution, recurrence of FAP related tumor, and/or to evaluate the response of a patient to a therapeutic treatment. In some embodiments, the methods are in vitro methods.

One embodiment is a method of treating a subject having FAP related cancer, the method of treatment comprising administering to the subject the FAP specific binding protein as described, optionally conjugated to a radioactive molecule and/or a cytotoxic agent, or as immunooncological agent. In various embodiments, the FAP binding protein as disclosed herein may be used in methods of treatment of FAP related cancer, optionally wherein the FAP binding protein is conjugated to a cytotoxic agent and/or to a radioactive molecule or expressed on the surface of target specific cancer associated fibroblast (CAF)-cells. For example, cancer cells might be solid tumor cells. Some embodiments relate to the use of the protein comprising the FAP binding protein labelled with a suitable radioisotope or cytotoxic compound in method for the treatment of FAP related tumors, in particular to control or kill FAP related tumor cells, for example malignant cells. In one embodiment, curative doses of radiation are selectively delivered of to FAP related tumor cells.

In some embodiments, methods for the treatment of FAP related diseases comprise the FAP binding protein as described herein or a fusion protein comprising the FAP binding protein as disclosed herein. In some embodiments, the treatment of FAP related diseases comprise FAP binding proteins as disclosed herein and further components to promote an immune response. As described herein, a FAP related cancer may be any of breast cancer, colorectal cancer, pancreatic cancer, lung cancer, brain cancer, intrahepatic bile duct, epithelial cell cancer, squamous cell carcinoma, and ovarian cancers, or tumors that are derived from non-epithelial tissues, such as melanoma and myeloma.

Compositions. Various embodiments relate to a composition comprising the FAP binding protein as described herein or a fusion protein comprising the FAP binding protein as disclosed herein. A composition comprising the protein comprising the FAP binding protein as defined above for use in medicine, preferably for use in the diagnosis or treatment of FAP related cancer. Compositions comprising the FAP binding protein as described herein or a fusion protein comprising the FAP binding protein as described above may be used in methods for diagnosis and/or treatment (including imaging) of FAP related diseases. In particular, compositions comprising the FAP binding protein as described above may be used for in methods for imaging, monitoring, and eliminating or inactivating pathological cells that express FAP.

Various embodiments relate to a diagnostic composition for the diagnosis of FAP related cancer comprising the FAP binding protein as defined herein and a diagnostically acceptable carrier and/or diluent. These include for example but are not limited to stabilizing agents, surface-active agents, salts, buffers, coloring agents etc. The compositions can be in the form of a liquid preparation, a lyophilisate, granules, in the form of an emulsion or a liposomal preparation.

The diagnostic composition comprising the FAP binding protein as described herein can be used for diagnosis of FAP related cancer, as described above.

Various embodiments relate to a pharmaceutical (e.g. therapeutical) composition for the treatment of diseases comprising the FAP binding protein as disclosed herein, and a pharmaceutically (e.g. therapeutically) acceptable carrier and/or diluent. The pharmaceutical (e.g. therapeutical) composition optionally may contain further auxiliary agents and excipients known per se. These include for example but are not limited to stabilizing agents, surface-active agents, salts, buffers, coloring agents etc. A therapeutical composition may comprise the FAP binding protein as disclosed herein and further agents that promote an immune response (immunooncology agent). The FAP specific binding protein as disclosed herein as part of a composition may counteract the inhibition of the immune response.

The pharmaceutical composition comprising the FAP binding protein as defined herein can be used for treatment of diseases, as described above.

The compositions contain an effective dose of the FAP binding protein as defined herein. The amount of protein to be administered depends on the organism, the type of disease, the age and weight of the patient and further factors known per se. Depending on the galenic preparation these compositions can be administered parenterally by injection or infusion, systemically, intraperitoneally, intramuscularly, subcutaneously, transdermally, or by other conventionally employed methods of application.

The composition can be in the form of a liquid preparation, a lyophilisate, a cream, a lotion for topical application, an aerosol, in the form of powders, granules, in the form of an emulsion or a liposomal preparation. The type of preparation depends on the type of disease, the route of administration, the severity of the disease, the patient and other factors known to those skilled in the art of medicine.

The various components of the composition may be packaged as a kit with instructions for use.

Preparation of FAP binding proteins. FAP binding proteins as described herein may be prepared by any of the many conventional and well known techniques such as plain organic synthetic strategies, solid phase-assisted synthesis techniques, fragment ligation techniques or by commercially available automated synthesizers. On the other hand, they may also be prepared by conventional recombinant techniques alone or in combination with conventional synthetic techniques. Furthermore, they may also be prepared by cell-free in vitro transcription/translation. Various embodiments relate to a polynucleotide encoding a FAP binding protein as disclosed herein. One embodiment further provides an expression vector comprising said polynucleotide, and a host cell comprising said isolated polynucleotide or the expression vector.

Various embodiments relate to a method for the production of a FAP binding protein as disclosed herein comprising culturing of a host cell under suitable conditions which allow expression of said FAP binding protein and optionally isolating said FAP binding protein.

For example, one or more polynucleotides which encode for the FAP binding protein may be expressed in a suitable host and the produced FAP binding protein can be isolated. A host cell comprises said nucleic acid molecule or vector. Suitable host cells include prokaryotes or eukaryotes. A vector means any molecule or entity (e.g., nucleic acid, plasmid, bacteriophage or virus) that can be used to transfer protein coding information into a host cell. Various cell culture systems, for example but not limited to mammalian, yeast, plant, or insect, can also be employed to express recombinant proteins. Suitable conditions for culturing prokaryotic or eukaryotic host cells are well known to the person skilled in the art. Cultivation of cells and protein expression for the purpose of protein production can be performed at any scale, starting from small volume shaker flasks to large fermenters, applying technologies well-known to any skilled in the art.

One embodiment is directed to a method for the preparation of a binding protein as detailed above, said method comprising the following steps: (a) preparing a nucleic acid encoding a FAP binding protein as defined herein; (b) introducing said nucleic acid into an expression vector; (c) introducing said expression vector into a host cell; (d) cultivating the host cell; (e) subjecting the host cell to culturing conditions under which a FAP binding protein is expressed, thereby producing a FAP binding protein as defined herein; (f) optionally isolating the FAP binding protein produced in step (e); and (g) optionally conjugating the FAP binding protein with further functional moieties as defined herein.

In general, isolation of purified FAP binding protein from the cultivation mixture can be performed applying conventional methods and technologies well known in the art, such as centrifugation, precipitation, flocculation, different embodiments of chromatography, filtration, dialysis, concentration and combinations thereof, and others. Chromatographic methods are well-known in the art and comprise without limitation ion exchange chromatography, gel filtration chromatography (size exclusion chromatography), hydrophobic interaction chromatography or affinity chromatography.

For simplified purification, the FAP binding protein can be fused to other peptide sequences having an increased affinity to separation materials. Preferably, such fusions are selected that do not have a detrimental effect on the functionality of the FAP binding protein or can be separated after the purification due to the introduction of specific protease cleavage sites. Such methods are also known to those skilled in the art.

Method of detection FAP. Some embodiments relate to a method of detecting (human) FAP in a sample, comprising contacting the sample with a FAP binding protein of the invention as described herein. In preferred embodiments, the sample is a sample obtained from a subject, wherein the subject preferably is a human subject. In preferred embodiments, the sample is, without being limited thereto, any one of blood, plasma, serum, tissue, tissue fluid, and urine. In preferred embodiments, the sample is, or is obtained from, a cancer tissue or cancer biopsy, wherein the cancer is a FAP-related cancer as described elsewhere herein.

### EXAMPLES

The following Examples are provided for further illustration of the invention. The invention is particularly exemplified by modifications of ubiquitin resulting in binding to FAP. The invention, however, is not limited thereto, and the following Examples merely show the practicability of the invention on the basis of the above description.

### Example 1: Mammalian Expression of Target

Expi293-F-cells were cultured with 0.5-1 Mio cells/ml in Expi293-™ Expression medium (Fisher scientific, 13469756) in shake flasks with 135 rpm, at 37° C., 8% CO₂ and 95% humidity. One day before transfection, cells were seeded with a density of 2.0 Mio cells/ml. On the day of transfection, cells were seeded with a density of 2.5 Mio/ml. 1 μg plasmid-DNA of hFAP-Fc, hCD26-Fc, mCD26-Fc per ml of culture volume or 0.5 μg hFAP-AviHis and 0.5 μg hBirA per ml of culture volume for biotinylated hFAP-His or 0.5 μg mFAP-AviHis and 0.5 μg hBirA per ml of culture volume for biotinylated mFAP-His were used and diluted in Opti-MEM I Reduced Serum Medium (Life Technologies, 31985-062). ExpiFectamine (Thermo Fisher, A14524) was diluted in Opti-MEM I Reduced Serum Medium, according to manufacturer information and incubated for 5 min at rt. Subsequently, the DNA-solution was added to the ExpiFectamine-mixture and incubated for 20 min at rt, before it was added to the cells. For protein expression cells were incubated at 37° C., 8% CO₂ and 95% humidity. After 16 h Enhancer (Thermo Fisher, A14524) was added to the transfection mixture. Supernatant was collected after 96-120 h, centrifuged and filtered through a 0.45 μm membrane.

For production of mFAP-Fc, ExpiCHO-cells were cultured in ExpiCHO Expression Medium (Thermo Fisher Scientific, A2910001) with 0.5 Mio cells/ml (see above). Cells were seeded with a density of 4.0 Mio/ml one day before transfection. For transfection, cells were seeded with 6.0 Mio/ml. 1 μg plasmid-DNA of mFAP-Fc per ml of culture volume was diluted in OptiPRO SFM (Thermo Fisher Scientific, 12309019). ExpiFectamine (Thermo Fisher, A29129) was diluted in OptiPRO SFM and mixed with the DNA-solution. After 3 min of incubation at rt, transfection mix was added to the cells. Cells were incubated for 24 h at 37° C. After incubation for 24 h at 37° C., ExpiFectamine Enhancer and ExpiFectamine Feed were added and cells were incubated at 32° C. Supernatant was collected after 120 h, centrifuged and filtered through a 0.45 μm membrane.

### Example 2. Identification of FAP Binding Proteins

Library construction and cloning of libraries. Different proprietary libraries comprising randomized amino acid positions in ubiquitin and/or inserts were in house synthesized by randomized oligonucleotides generated by synthetic trinucleotide phosphoramidites (ELLA Biotech) to achieve a well-balanced amino acid distribution with simultaneously exclusion of cysteine and other amino acid residues at randomized positions.

The corresponding cDNA libraries were amplified by PCR and ligated with a modified pCD87SA phagemid (herein referred to as pCD12) using standard methods known to a skilled person. The pCD12 phagemid comprises a modified torA leader sequence (deletion of amino acid sequence QPAMA) to achieve protein processing without additional amino acids at the N terminus. Aliquots of the ligation mixture were used for electroporation of E. coli ER2738 (Lucigen). Unless otherwise indicated, established recombinant genetic methods were used.

Target. The selection was performed with the extracellular domain of human FAP protein and/or the extracellular domain of mouse FAP protein as target. On the one hand, IgG₁-Fc-fused proteins were applied, on the other hand biotinylated AviHis-fused proteins were applied (in house cloned and expressed as described in Example 1).

Primary selection by TAT Phage Display. The naive libraries were enriched against FAP using phage display as selection system. After transformation of competent bacterial ER2738 cells (Lucigene) with phagemid pCD12 carrying the library, phage amplification and purification was carried out using standard methods known to a skilled person. For selection the target protein was immobilized on magnetic beads. Target proteins fused to IgG₁-Fc were immobilized on Protein A Dynabeads. Site-directed biotinylated target proteins fused to an AviHis-tag were immobilized on M-270 Epoxy Dynabeads. The FAP concentration during phage incubation was lowered from 140 nM (first round) to 20 nM (third round) or 5 nM (fourth round) for the biotinylated AviHis-fused target proteins including a target switch in each round between human FAP and mouse FAP, starting with human FAP. The FAP concentration for the IgG₁-Fc-fused target was lowered from 100 nM (first round) to 20 nM (third round) or 5 nM (fourth round) only using human FAP.

The selection against the IgG₁-Fc-fused target was performed with three to four rounds, depending on the library and included a preselection with gammanorm (Octopharma, Cat. No. PZN 13336380) on Protein A Dynabeads in round 2 and off-target extracellular domain of human CD26 fused to IgG₁-Fc (AcroBiosystems, Cat. No. DP4-H5266) on Protein A Dynabeads in round 3 and 4. An additional preincubation of the phage particles in mouse serum for 23 h at 37° C. before round 3 and 4 was performed.

The selection against the biotinylated AviHis-fused targets was also performed with three to four rounds, depending on the library and included a preselection only with human CD26 fused to IgG₁-Fc (AcroBiosystems, Cat. No. DP4-H5266) on Protein A Dynabeads starting in round 3.

All selection rounds were performed with the automated KingFisher-System (Thermo Fisher) to isolate, wash and capture the desired phage-target complexes on the magnetic beads. FAP bound phages were eluted by trypsin.

To identify target specific phage pools, eluted and reamplified phages of each selection round were analysed by phage pool ELISA. Wells of medium binding microtiter plates (Greiner Bio-One) were coated with human FAP (2.5 μg/ml) or mouse FAP (2.5 μg/ml), gammanorm (2.5 μg/ml), and human CD26 (2.5 μg/ml). Biotinylated AviHis-fused FAP was coated via Streptavidin. Bound phages were detected using α-M13 HRP-conjugated antibody (GE Healthcare).

Cloning of target binding phage pools into an expression vector. Selection pools showing specific binding to FAP in phage pool ELISA were amplified by PCR according to methods known in the art, cut with appropriate restriction nucleases and ligated into a derivative of the expression vector pET-28a (Merck, Germany) comprising a Strep-Tag II (IBA GmbH).

Single colony hit analysis. After transformation of BL21 (DE3) cells, kanamycin-resistant single colonies were picked automatically using a Qpix2 colony picker. Expression of the FAP-binding proteins was achieved by cultivation in 384-well plates (Greiner Bio-One) using auto induction medium (Studier, 2005, Protein Expr. Purif. 41 (1): 207-234). Cells were harvested and subsequently lysed mechanically by freeze/thaw cycles. After centrifugation the resulting supernatants were initially screened by ELISA with immobilized ProtA/FAP-Fc on high binding 384 ELISA microtiter plates (Greiner Bio-One). The protein bound to FAP-Fc was detected by Strep-Tactin HRP Conjugate (IBA GmbH) in combination with TMB-Plus substrate (Biotrend, Germany). The reaction was stopped by addition of 0.2 M H₂SO₄ solution and measured in a plate reader at 450 nm versus 620 nm. In a confirmation screen, the binding of the initial hits was analysed vs FAP-Fc (ON-target) and IgG-Fc (OFF-target). Specific hits were selected for μ-scale purification, SPR analysis, sequencing and further for expression and analytics in lab scale.

### Example 3A: Purification of Target Molecules

Cell culture supernatant of hFAP-Fc-His, mFAP-Fc-His, hCD26-Fc-His, and mCD26-Fc-His expressions was centrifuged and filtrated for application to affinity chromatography on a His Trap excel 1 mL column or HiTrap Protein A HP 5 ml (Cytiva, buffer conditions according to manufacturer instructions). Cell culture supernatant of hFAP-Avi-His, mFAP-Avi-His, hCD26-Avi-His and mCD26-Avi-His expressions was centrifuged and filtrated for application to affinity chromatography on a His Trap excel 1 mL column (Cytiva, buffer conditions according to manufacturer instructions).

The eluted target proteins were applied to a Superdex 200 XK 16/600 gel filtration column. The purity of the recovered target protein was analyzed and confirmed by SDS-PAGE and SE-HPLC. The enzymatic activity was confirmed by a fluorogenic assay based on the ability of the FAP target proteins to convert the substrate benzyloxycarbonyl-Gly-Pro-7-amido-4-methylcoumarin (Z-GP-AMC) to 7-amino-4-methylcoumarin (AMC) and of the CD26 off-targets to convert H-Gly-Pro-7-amino-4-methylcoumarin (GP-AMC) to AMC.

### Example 3B: Enzymatic Activity Test of hFAP-Fc-his in the Presence of Affilin® Proteins

To investigate if Affilin® proteins influence the enzymatic activity of hFAP-Fc-His the activity assay was performed as described in Example 3A but in the presence of Affilin® proteins. Affilin®-217990 (SEQ ID NO: 1), Affilin®-217862 (SEQ ID NO: 3), Affilin®-217832 (SEQ ID NO: 4), Affilin®-217917 (SEQ ID NO: 6), Affilin®-217993 (SEQ ID NO: 11), Affilin®-219750 (SEQ ID NO: 15), Affilin®-219235 (SEQ ID NO: 17), Affilin®-220134 (SEQ ID NO: 18), Affilin®-220164 (SEQ ID NO: 19), and Affilin®-220257 (SEQ ID NO: 22) were tested. The reaction mixture contained 0.9 nM hFAP-Fc-His and 1 μM Affilin® protein.

None of the tested Affilin® protein decreased the enzymatic activity of hFAP-Fc-His.

### Example 4. Expression and Purification of FAP Binding Proteins

The genes for FAP binding proteins were cloned into an expression vector using standard methods known to a skilled person, purified and analyzed as described below. All FAP specific proteins were expressed and highly purified by affinity chromatography and gel filtration. After affinity chromatography using a Strep-Tactin Superflow high capacity column the eluted proteins were applied to a size exclusion chromatography (Superdex 75 HiLoad 16/600 or Sephacryl S200HR 16/600 column) using an ÄKTA xpress system (GE Healthcare). Elution with PBS containing 500 mM NaCl pH 7.4 was carried out in three column volumes. Following SDS-PAGE analysis positive fractions were pooled and their protein concentrations were measured.

Further analysis included SDS-PAGE, RP-HPLC and SE-HPLC. Protein concentrations were determined by absorbance measurement at 280 nm using the specific molar absorbent coefficient. Reversed phase chromatography (RP-HPLC) was performed using an Ultimate 3000 HPLC system (Thermo Fisher Scientific) and a PLRP-S(5 μm, 300 Å) column (Agilent). The purity resulted in >78%. Analytic size exclusion chromatography (SE-HPLC) was performed using an Ultimate 3000 HPLC system (Thermo Fisher Scientific) and a Superdex75 increase 5/150 GL (Cytiva). No aggregation was detected.

### Example 5. Analysis of FAP Binding Proteins (Surface Plasmon Resonance, SPR)

Recombinant protein A was immobilized on a High Capacity Amine sensor chip (Bruker) after NHS/EDC activation resulting in approx. 2000 RU with a Sierra SPR-32 system (Bruker). The chip was equilibrated with SPR running buffer (PBS 0.05%, Tween pH 7.3). Injection of ethanolamine after ligand immobilization was used to block unreacted NHS groups. The Fc-tagged FAP and CD26 target molecules were injected with 60 nM followed by the injection of FAP binding proteins. Upon binding, target analyte was accumulated on the surface increasing the refractive index. This change in the refractive index was measured in real time and plotted as response or resonance units versus time. The FAP binding proteins were applied to the chip in serial dilutions with a flow rate of 30 μl/min. The association was performed for 120 seconds and the dissociation for 180 seconds. After each run, the chip surface was regenerated with 30 μl regeneration buffer (10 mM glycine pH 2.0) and equilibrated with running buffer. Binding studies were carried out by the use of the Sierra SPR-32 system (Bruker); data evaluation was operated via the Sierra Analyser software, provided by the manufacturer, by the use of the Langmuir 1:1 model (RI=0). Evaluated dissociation constants (K_{D}) were standardized against the immobilized protein and indicated. Table 1 shows the binding affinity of FAP binding proteins to hFAP.

TABLE 1
Binding affinity (K_{D}) of FAP binding proteins vs. human FAP

| SEQ ID NO: | Affilin ® | K_{D} vs. hFAP-Fc |
|---|---|---|
| 1 | 217990 | 14 nM |
| 2 | 217966 | 7.8 nM |
| 15 | 219750 | 6.0 nM |
| 16 | 220198 | 3.3 nM |
| 3 | 217862 | 7.0 nM |
| 4 | 217832 | 3.2 nM |
| 5 | 217863 | 1.9 nM |
| 24 | 223054 | 2.0 nM |
| 25 | 223019 | 2.0 nM |
| 6 | 217917 | 43 nM |
| 17 | 219235 | 5.1 nM |
| 19 | 220164 | 3.8 nM |
| 18 | 220134 | 1.7 nM |
| 26 | 223078 | 2.0 nM |
| 27 | 223077 | 3.0 nM |
| 11 | 217993 | 23 nM |
| 22 | 220257 | 3.6 nM |

Affilin®-217990 (SEQ ID NO: 1), Affilin®-217862 (SEQ ID NO: 3), Affilin®-217832 (SEQ ID NO: 4), Affilin®-217863 (SEQ ID NO: 5), Affilin®-219235 (SEQ ID NO: 17), Affilin®-220164 (SEQ ID NO: 19), Affilin®-223078 (SEQ ID NO: 26), and Affilin®-223077 (SEQ ID NO: 27) bind (cross-specific) to mouse FAP (mFAP) and human FAP (hFAP). The affinity of Affilin®-220164 vs. mFAP is 3.1 nM. The affinity of Affilin®-223078 vs. mFAP is 3 nM. The affinity of Affilin®-223077 vs. mFAP is 5 nM. The affinity of Affilin®-219235 vs. mFAP is 21.6 nM. The affinity of Affilin®-217990 vs. mFAP is 90 nM.

Affilin®-217990 and Affilin®-217832, respectively, bind cross-specific to cynomolgus FAP (cFAP) and human FAP (hFAP).

No FAP specific Affilin® as disclosed herein binds to hCD26 or to mCD26.

### Example 6. Competition of Binding Proteins for Epitopes

Competitive binding of two isolated Affilin® proteins to hFAP-Fc was investigated as followes: the first Affilin®-protein was immobilized on a CM5 Biacore chip (˜200 RU) using NHS/EDC chemistry. 250 nM hFAP-Fc were injected with or without four-fold excess of the second Affilin® protein. Results are shown in Table 2. In Table 2, “competition” means that the binding of the first Affilin® was influenced by the presence of the second Affilin®, and vice versa. Affilin®-217993, Affilin®-217990, and Affilin®-217832 bind to the same or overlapping epitopes, i.e. to the same or overlapping surface exposed amino acids.

TABLE 2
Affilin ® proteins bind to the same or overlapping epitope

| | | 1 | 4 | 11 |
|---|---|---|---|---|
| SEQ ID NO: | Affilin ®- | 217990 | 217832 | 217993 |
| 1 | 217990 | — | competition | competition |
| 4 | 217832 | competition | — | competition |
| 11 | 217993 | competition | competition | — |

### Example 7. Functional Characterization: FAP Binding Proteins are Stable at High Temperatures

Thermal stability of the FAP specific Affilin® proteins was determined by differential scanning fluorimetry (DSF) or circular dichroism (CD). For DSF measurements each probe was transferred at concentrations of 0.25 μg/μL to a LightCycler 480 Multiwell Plate 96 (Roche), and SYPRO Orange dye was added at a suitable dilution. A temperature ramp from 20 to 90° C. was programmed with a heating rate of 1° C./min (LightCycler480 RT-PCR-System, Roche). Fluorescence was constantly measured at an excitation wavelength of 465 nm and the emission wavelength at 580 nm. For CD measurements the samples were desalted in 20 mM NaH₂PO₄ PH 7.0 using a HiTrap Desalting 5 ml column (Cytiva). FAP binding proteins were diluted to a concentration of 0.2-0.4 μg/μl. A temperature ramp from 20 to 90° C. was run with a heating rate of 0.5° C./min (J-815, Jasco).

The midpoints of transition for the thermal unfolding (Tₘ, melting points) were determined and are shown in Table 3.

TABLE 3
FAP binding proteins are stable at high temperatures

| SEQ ID NO: | Affilin ® | Tₘ |
|---|---|---|
| 1 | 217990 | >80° C. |
| 2 | 217966 | 78° C. |
| 3 | 217862 | 72° C. |
| 4 | 217832 | 80° C. |
| 5 | 217863 | >80° C. |

### Example 8. Functional Characterization: Specific Binding to Cell Surface Expressed hFAP (Flow Cytometry)

Flow cytometry was used to analyze the interaction of FAP binding proteins with cell surface-exposed hFAP and mFAP. FAP overexpressing human embryonic kidney cell line HEK293, CD26-overexpressing HEK293-cells, native FAP expressing cell line Wi38 and empty vector control HEK293-pEntry cells were used. The anti-hFAP antibody (R&D Systems, MAB3715-100) with a concentration of 1 μg/ml in combination with anti-mouse-IgG-Alexa488 (Invitrogen, A10680) with a 1:1000 dilution was used as positive control for hFAP-expressing cells. Anti-mFAP antibody (R&D Systems, MAB9727) with a concentration of 1 μg/ml in combination with anti-rat-IgG-Alexa488 (Invitrogen, A11006) with a dilution of 1:1000 was used as positive control for mFAP-expressing cells.

Cells were trypsinized and resupended in medium containing FCS, washed and stained in pre-cooled FACS blocking buffer (3% FCS/PBS). A suspension with a cell concentration of 1×10⁶ cells/ml was prepared for cell staining and filled with 100 μl/well into a 96 well plate (Greiner) in triplicate for each cell line.

Affilin® proteins were tested with a concentration of 1 μM, 100 nM, 10 nM, or 1 nM on FAP expressing cells HEK293-hFAP, HEK293-mFAP and Wi38-cells. To exclude an unspecific binding, Affilin® proteins were also incubated on CD26 expressing cells HEK293-hCD26 and HEK293-mCD26 and control cells HEK293-pEntry with the same concentrations. Comparable amounts of wildtype ubiquitin (clone 139090) were used as negative control. Supernatants were removed after 45 min, cells were washed in blocking buffer and 100 μl/well rabbit anti-StrepTag antibody (GenScript; A00626), 1:300 diluted in FACS blocking buffer were added. After removal of the primary antibody, goat anti-rabbit-IgG-Alexa Fluor 488 antibody (Invitrogen; A11008) was applied in a 1:1000 dilution. Flow cytometry measurement was conducted on the Guava easyCyte 5HT device from Merck-Millipore at excitation wavelength 488 nm and emission wavelength 525/30 nm.

The FACS experiments show that all Affilin® proteins disclosed herein bind to hFAP expressed on cell surfaces of HEK293 cells. A particular strong binding (+++) was observed for Affilin®. 217863, Affilin®-217862, Affilin®-217832, Affilin®-220164, Affilin®-223078, Affilin®-223077, Affilin®-223019, Affilin®-220257, and Affilin®-220198 (see Table 4).

For Affilin®-217990, Affilin®-220164, Affilin®-223078, Affilin®-223077, and Affilin®-219235 a binding on hFAP expressing cell line HEK293-hFAP and mFAP-expressing cell line HEK293-mFAP could be confirmed. No binding to control cells HEK293-hCD26-cells, HEK293-mCD26-cells or HEK293-pEntry was observed for FAP binding Affilin® proteins.

Further, all Affilin® proteins disclosed herein showed binding on native hFAP-expressing Wi38-cells.

Anti-FAP-antibodies show a positive staining on all FAP-expressing cells.

Wildtype ubiquitin showed no binding on FAP-expressing cells.

TABLE 4
Binding of Affilin ® proteins (1 μM) on HEK293-hFAP-cells

| Affilin ® | SEQ ID NO: | HEK293-hFAP |
|---|---|---|
| 217863 | 5 | +++ |
| 217862 | 3 | +++ |
| 217832 | 4 | +++ |
| 220164 | 19 | +++ |
| 223019 | 25 | +++ |
| 223078 | 26 | +++ |
| 223077 | 27 | +++ |
| 220257 | 22 | +++ |
| 220198 | 16 | +++ |
| 219750 | 15 | ++(+) |
| 220134 | 18 | ++(+) |
| 223054 | 24 | ++ |
| 219235 | 17 | ++ |
| 217990 | 1 | ++ |
| 217966 | 2 | ++ |
| 217993 | 11 | + |
| 217895 | 12 | + |
| Ubiquitin | 13 | no binding |

### Example 9. Binding Affinity to FAP in Serum after Long-Term Incubation (ELISA)

High binding plates (Greiner, 781061) were immobilized with 2.5 μg/ml hFAP-Fc over night at 4° C. Dilution series of 3 UM to 0.07 PM of Affilin® proteins were incubated in 100% human serum or 100% mouse serum for 24 h at 37° C.

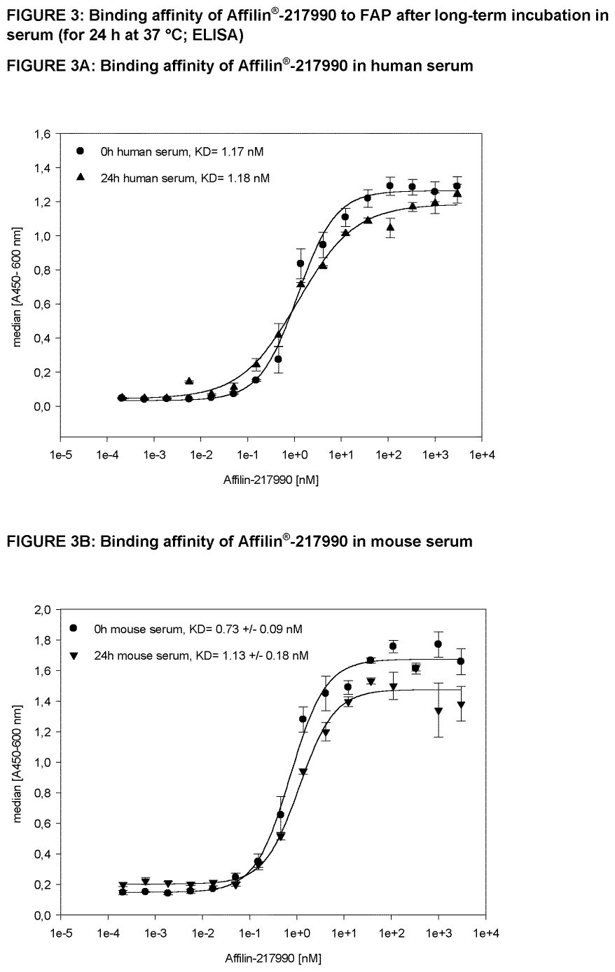

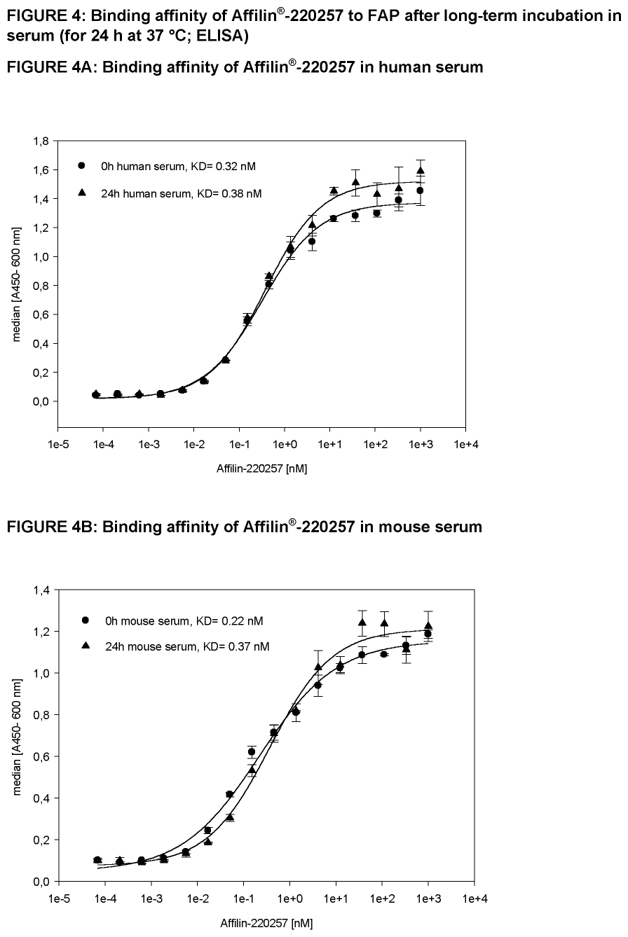

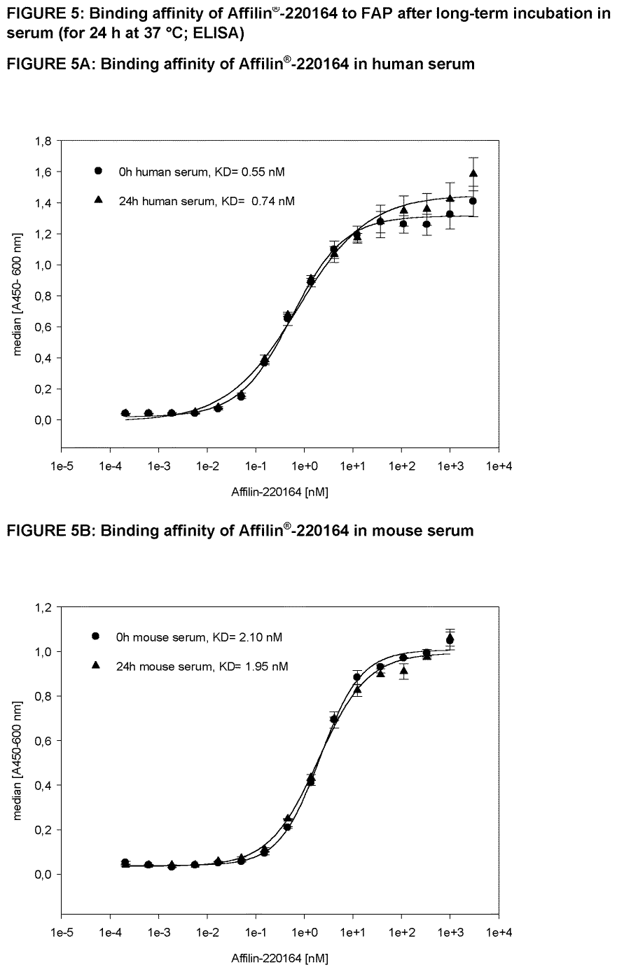

ELISA-plates were washed three times with PBST (PBS+ 0.1% Tween) and blocked with 3% BSA/0.5% Tween/PBS 1 h at rt. After preincubation for 0 h or 24 h in the presence of serum the dilution series were incubated on ELISA-plates for 30 min at room temperature (rt). Wells were washed with PBST and incubated with biotinylated anti-ubiquitin-antibody (1:300) for 30 min at rt. The binding was visualized with Streptavidin-HRP (1:5.000). Affilin® proteins show no significant change in K_{D} after 24 h incubation in human or mouse serum (see FIGS. 3-5). K_{D}-values are summarized in Table 5. ELISA analysis confirmed the high stability of FAP binding proteins in serum.

TABLE 5
High affinity binding of Affilin ®-proteins to
mouse FAP (mFAP) and human FAP (hFAP) in serum

| | | Mouse serum vs | | Human serum vs | |
|---|---|---|---|---|---|
| | | hFAP-Fc K_{D} [nM] | | hFAP-Fc K_{D} [nM] | |
| CID | SEQ ID NO: | 0 h | 24 h | 0 h | 24 h |
| 217990 | 1 | 0.7 | 1.1 | 1.2 | 1.2 |
| 217832 | 4 | 0.3 | 0.7 | 0.4 | 0.5 |
| 219750 | 15 | 0.4 | 0.5 | 0.3 | 0.4 |
| 220198 | 16 | 1.5 | 1.9 | 0.5 | 0.4 |
| 220134 | 18 | 0.6 | 0.7 | 0.6 | 0.6 |
| 220164 | 19 | 2.1 | 2.0 | 0.6 | 0.8 |
| 220257 | 22 | 0.2 | 0.4 | 0.3 | 0.4 |

### Example 10. Binding Affinity to FAP in Serum after Long-Term Incubation (Cell Binding Assay-Flow Cytometry)

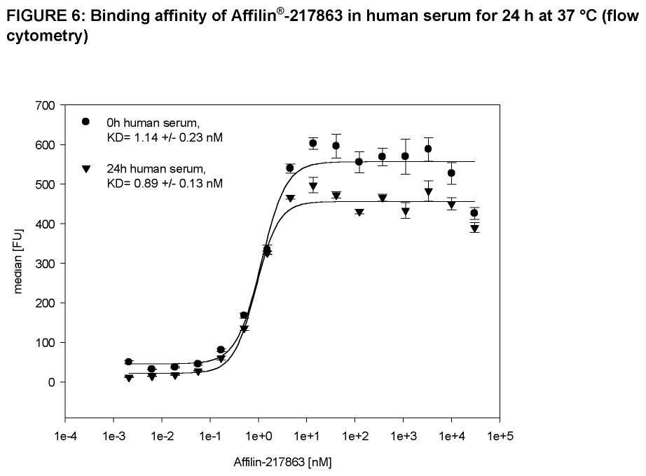

Dilution series from 30 UM to 2.1 μM of Affilin®-217966 (SEQ ID NO: 2), Affilin®-217862 (SEQ ID NO: 3), and Affilin®-217863 (SEQ ID NO: 5) were incubated in 100% human serum for 24 h at 37° C. hFAP expressing HEK293-cells were thawed, washed with medium containing FCS, subsequently washed with FACS blocking buffer (PBS/0.1% Sodium Azide/3% FCS) and 100 μl seeded in 96-well round bottom plates with a density of 1×10⁶ cells/ml. Dilution series of Affilin proteins were incubated with human serum for 24 h and 0 h (control) at 37° C. HEK293-hFAP-cells were incubated with dilution series for 45 min at 4° C. Cells were centrifuged and supernatants were removed. Cells were washed with FACS blocking buffer and 100 μl/well rabbit anti-Strep-Tag antibody (GenScript; A00626), 1:300 diluted in FACS blocking buffer were added. After removal of the primary antibody goat anti-rabbit IgG Alexa Fluor 488 antibody (Invitrogen; A11008) was applied in a 1:1000 dilution in FACS blocking buffer. Flow cytometry measurement was conducted on the Guava easyCyte 5HT device from Merck-Millipore at excitation wavelength 488 nm and emission wavelength 525/30 nm. Results are shown in FIG. 6. The K_{D}-determination of Affilin®-217863 shows no significant difference in binding to FAP even after 24 h serum incubation. Affilin®-217863 is stable in human serum. Similar results were obtained with Affilin®-217862 and Affilin®-217966.

1. A protein comprising an amino acid sequence of at least 90% identity to any one of SEQ ID NOs: 1-12 and 15-27, wherein the protein has a specific binding affinity for human Fibroblast Activation Protein (hFAP) of less than 50 nM.

2. The protein of claim 1, wherein the protein is a multimer.

3. The protein of claim 1, wherein the protein is a fusion protein.

4. The protein of claim 1, wherein the protein further comprises one or more coupling sites for coupling of one or more chemical moieties.

5. The protein of claim 1, wherein the protein further comprises at least one diagnostically active moiety.

6. The protein of claim 5, wherein the diagnostically active moiety is selected from the group consisting of a radionuclide, a fluorescent protein, a photosensitizer, a dye, an enzyme, one or more magnetic beads, one or more metallic beads, one or more colloidal particles, an electron-dense reagent, biotin, digoxigenin, a hapten, a CAR-T, and one or more exosomes, or any combination thereof.

7. The protein of claim [ ], wherein the protein further comprises at least one therapeutically active moiety.

8. The protein of claim 7, wherein the therapeutically active moiety is selected from the group consisting of a monoclonal antibody or a fragment thereof, a binding protein, a receptor or receptor domain, a receptor ligand, a radionuclide, a cytotoxic compound, a cytokine, a chemokine, an enzyme, a CAR-T, one or more exosomes, and derivatives thereof, or any combination thereof.

9. The protein of claim 1, wherein the protein comprises additionally at least one moiety modulating pharmacokinetics.

10. (canceled)

11. (canceled)

12. A method for producing the protein of claim 1, the method comprising:
(a) culturing a host cell comprising an expression vector encoding the protein of claim 1 under conditions suitable to produce said protein; and
(b) isolating said protein produced from the host cell and/or from the medium in which the host cell is cultured.

13. A method for detecting FAP present in a sample, the method comprising:
(a) contacting the sample with a composition comprising the protein of claim 1 to form a complex; and
(b) detecting the complex,
whereby FAP present in the sample is detected.

14. A method for treatment or diagnosis of a disease, disorder, or condition associated with FAP expression in a subject in need thereof, the method comprising contacting a biological sample present in or isolated from the subject with a composition comprising the protein of claim 1, optionally wherein the protein of claim 1 is conjugated to a therapeutically active moiety and/or a diagnostically active moiety.

15. The method of claim 14, wherein the disease, disorder, or condition is a tumor and/or a cancer associated with FAP expression, optionally wherein the tumor and/or the cancer is a tumor and/or a cancer of nonepithelial tissue, melanoma, myeloma, a breast tumor and/or cancer, a colorectal tumor and/or cancer, a pancreatic tumor and/or cancer, a lung tumor and/or cancer, a brain tumor and/or cancer, an intrahepatic bile duct tumor and/or cancer, and/or an ovarian tumor and/or cancer.

16. The method of claim 14, wherein the composition comprises and a pharmaceutically acceptable carrier and/or diluent.

17. The method of claim 1, wherein:
(i) the therapeutically active moiety is selected from the group consisting of a monoclonal antibody or a fragment thereof, a binding protein, a receptor or receptor domain, a receptor ligand, a radionuclide, a cytotoxic compound, a cytokine, a chemokine, an enzyme, a CAR-T, one or more exosomes, and derivatives thereof, or any combination thereof; and/or
(ii) the diagnostically active moiety selected from the group consisting of a radionuclide, a fluorescent protein, a photosensitizer, a dye, an enzyme, one or more magnetic beads, one or more metallic beads, one or more colloidal particles, an electron-dense reagent, biotin, digoxigenin, a hapten, a CAR-T, and one or more exosomes, or any combination thereof.
